(19) United States
(12) Patent Application Publication
(10) Pub. No.: US 2025/0221678 A1
(43) Pub. Date: Jul. 10, 2025

(54) SPARE ASSEMBLY FOR MEDICAL IMAGING SYSTEM, SUSPENSION APPARATUS, AND X-RAY IMAGING SYSTEM

(71) Applicant: GE Precision Healthcare LLC, Waukesha, WI (US)

(72) Inventors: Jianqiang Yang, Beijing (CN); Rongquan Yan, Beijing (CN)

(21) Appl. No.: 19/012,669

(22) Filed: Jan. 7, 2025

(30) Foreign Application Priority Data
Jan. 8, 2024 (CN) 202410032657.X

(51) Int. Cl. A61B 6/00

(52) U.S. Cl. CPC A61B 6/4476; A61B 6/4464

(57) ABSTRACT

The present application provides a spare assembly for a medical imaging system, a suspension apparatus, and an X-ray imaging system. The medical imaging system includes a driving assembly, and the driving assembly includes a rotating shaft. The spare assembly includes a worm wheel portion mounted on the rotating shaft and a worm portion mounted at a predetermined distance from the worm wheel portion. The worm portion has a first adjusting portion and a second adjusting portion. The first adjusting portion is operable to engage the worm portion with the worm wheel portion. The second adjusting portion is operable to drive the worm wheel portion by means of the worm portion, so as to drive the rotating shaft to move.

### CROSS-REFERENCE TO RELATED APPLICATIONS

This application is based on and claims priority to Chinese Patent Application No. 202410032657.X, filed on Jan. 8, 2024, the entire contents of which is herein incorporated by reference.

### TECHNICAL FIELD

The present invention relates to medical imaging technology, and relates more specifically to a spare assembly for a medical imaging system, a suspension apparatus, and an X-ray imaging system.

### BACKGROUND

In an X-ray imaging system, radiation from an X-ray source is emitted toward a subject, and the subject under examination is usually a patient in a medical diagnosis application. Some of the radiation passes through the subject under examination and impacts a detector, which is divided into a matrix of discrete elements (e.g., pixels). The detector elements are read to generate an output signal on the basis of the amount or intensity of radiation that impacts each pixel region. The signal can then be processed to generate a medical image that can be displayed for review, and the medical image can be displayed in a display apparatus of the X-ray imaging system.

X-ray imaging systems include suspended X-ray imaging systems, ground rail X-ray imaging systems, and movable X-ray imaging systems. In particular, for suspended X-ray imaging systems, a suspension apparatus is mounted on the ceiling, and includes guide rails, a telescopic cylinder, a tube assembly, etc. The suspension apparatus engages in five-axis movement, including three-axis translation and two-axis rotation. For the movement of each axis, a corresponding driving assembly needs to be provided. When any driving assembly or a component therein fails, the suspension apparatus immediately stops moving and remains in its current position, and needs to be checked and repaired by a field engineer. In particular, during the repair process, since components such as the telescopic cylinder and the tube assembly are all heavy (about 100 kg), a hoisting mechanism is required for removal, replacement, and reinstallation of the respective components. Moreover, during the entire process, the suspension apparatus can no longer move, and image capture can no longer be performed. The entire process is time-consuming and labor-consuming, and the image capture progress of the hospital is delayed.

### SUMMARY

The present invention provides a spare assembly for a medical imaging system, a suspension apparatus, and an X-ray imaging system.

The exemplary embodiments of the present invention provide a spare assembly for a medical imaging system. The medical imaging system comprises a driving assembly, the driving assembly comprising a rotating shaft, and the spare assembly comprising: a worm wheel portion mounted on the rotating shaft; and a worm portion mounted at a predetermined distance from the worm wheel portion, the worm portion having a first adjusting portion and a second adjusting portion, the first adjusting portion being operable to engage the worm portion with the worm wheel portion, and the second adjusting portion being operable to drive the worm wheel portion by means of the worm portion, so as to drive the rotating shaft to move.

The exemplary embodiments of the present invention provide a suspension apparatus. The suspension apparatus comprises a set of perpendicularly mounted guide rails, a sliding member, and a telescopic cylinder, the guide rails being mounted on a ceiling, the sliding member being mounted on the guide rails, the sliding member being connected to the telescopic cylinder, and the driving assembly being mounted within the sliding member to drive the telescopic cylinder to move. The driving assembly comprises a rotating shaft, and the spare assembly comprises: a worm wheel portion mounted on the rotating shaft; and a worm portion mounted at a predetermined distance from the worm wheel portion, the worm portion having a first adjusting portion and a second adjusting portion, the first adjusting portion being operable to engage the worm portion with the worm wheel portion, and the second adjusting portion being operable to drive the worm wheel portion by means of the worm portion, so as to drive the rotating shaft to move.

Exemplary embodiments of the present invention provide an X-ray imaging system. The X-ray imaging system comprises a suspension apparatus, and the suspension apparatus comprises a set of perpendicularly mounted guide rails, a sliding member, and a telescopic cylinder, the guide rails being mounted on a ceiling, the sliding member being mounted on the guide rails, the sliding member being connected to the telescopic cylinder, and the driving assembly being mounted within the sliding member to drive the telescopic cylinder to move. The driving assembly comprises a rotating shaft, and the spare assembly comprises: a worm wheel portion mounted on the rotating shaft; and a worm portion mounted at a predetermined distance from the worm wheel portion, the worm portion having a first adjusting portion and a second adjusting portion, the first adjusting portion being operable to engage the worm portion with the worm wheel portion, and the second adjusting portion being operable to drive the worm wheel portion by means of the worm portion, so as to drive the rotating shaft to move.

Other features and aspects will become apparent from the following detailed description, drawings, and claims.

### BRIEF DESCRIPTION OF THE DRAWINGS

The present invention can be better understood by means of the description of the exemplary embodiments of the present invention in conjunction with the drawings, in which:

FIG. 1 is a schematic diagram of an X-ray imaging system according to some embodiments of the present application;

FIG. 2 is a schematic diagram of a sliding member according to some embodiments of the present application;

FIG. 3 is a schematic diagram of a driving assembly and a spare assembly according to some embodiments of the present application;

FIG. 4 is a schematic diagram of the spare assembly in a first state according to some embodiments of the present application;

FIG. 5 is a schematic diagram of the spare assembly in a second state according to some embodiments of the present application;

FIG. 6 is a schematic diagram of a driving assembly according to some embodiments of the present application;

FIG. 7 is a schematic diagram of a wire rope fixing support in a first position according to some embodiments of the present application;

FIG. 8 is a schematic diagram of the wire rope fixing support in a second position according to some embodiments of the present application; and

FIG. 9 is a cross-sectional schematic diagram of a safety locking mechanism according to some embodiments of the present application.

### DETAILED DESCRIPTION

Specific embodiments of the present invention will be described below. It should be noted that in the specific description of said embodiments, for the sake of brevity and conciseness, the present description cannot describe all of the features of the actual embodiments in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first” and “second” and similar terms used in the description and claims of the patent application of the present invention do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

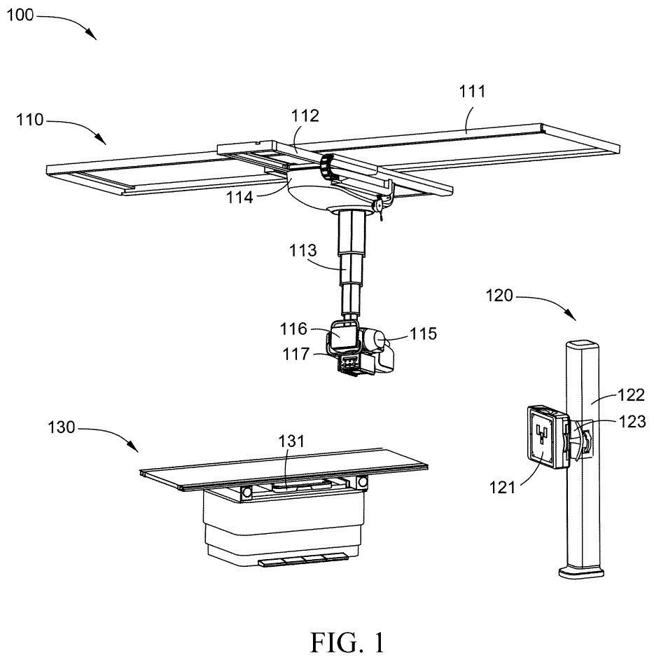

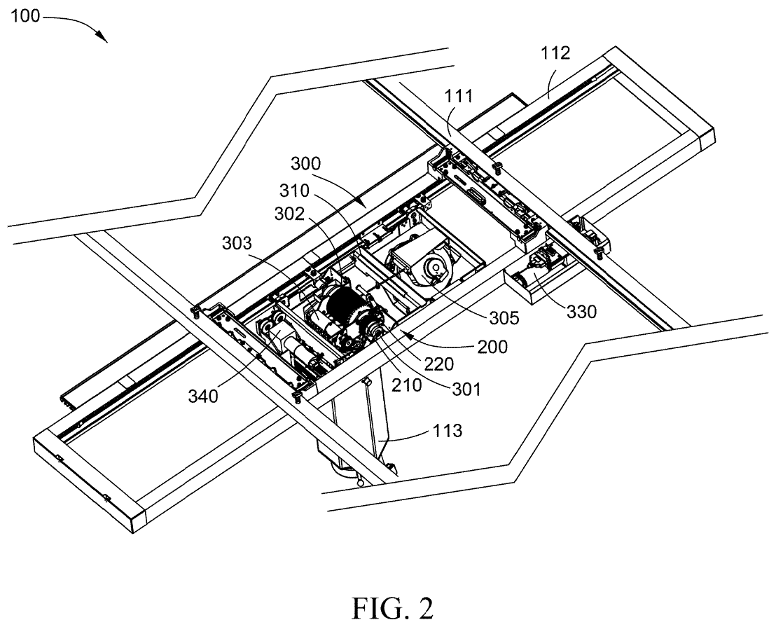

FIG. 1 shows an X-ray imaging system 100 according to some embodiments of the present invention, and FIG. 2 shows a schematic diagram of a sliding member of a suspension apparatus according to some embodiments of the present invention. As shown in FIGS. 1 and 2, the X-ray imaging system 100 includes a suspension apparatus 110, a wall stand apparatus 120, and an examination table apparatus 130. The suspension apparatus 110 includes a set of perpendicularly mounted guide rails, a telescopic cylinder 113, a sliding member 114, and a tube assembly 115.

For ease of description, in the present application, the x-axis, y-axis, and z-axis are defined as the x-axis and y-axis being located in the horizontal plane and perpendicular to one another, and the z-axis being perpendicular to the horizontal plane. Specifically, the direction in which a longitudinal guide rail 111 is located is defined as the x-axis, the direction in which a transverse guide rail 112 is located is defined as the y-axis direction, and the direction of extension of the telescopic cylinder 113 is defined as the z-axis direction, and the z-axis direction is the vertical direction.

The set of guide rails includes the longitudinal guide rail 111 and the transverse guide rail 112 that are perpendicularly arranged, wherein the longitudinal guide rail 111 is mounted on the ceiling and the transverse guide rail 112 is mounted on the longitudinal guide rail 111. The sliding member 114 is arranged between the transverse guide rail 112 and the telescopic cylinder 113, and the telescopic cylinder 113 is used to carry the tube assembly 115. The telescopic cylinder 113 includes a plurality of cylinders having different inner diameters, and the plurality of cylinders can be sleeved, sequentially from bottom to top, in the cylinder located thereabove, thereby achieving telescoping, and the telescopic cylinder 113 can be telescopic (or movable) in the vertical direction, i.e., the telescopic cylinder 113 can drive the tube assembly to move along the z-axis direction.

The tube assembly 115 includes an X-ray tube, and the X-ray tube may produce X-rays and project the X-rays to an intended region of interest (ROI) of a patient. Specifically, the X-ray tube may be positioned adjacent to a beam limiter 117, and the beam limiter 117 is used to align X-rays with an intended region of interest of the patient. At least part of the X-rays may be attenuated through the patient, and may be incident on a detector 121/131.

The suspension apparatus 110 further includes a tube console 116. The tube console 116 is mounted on the tube assembly. The tube console 116 includes user interfaces such as a display screen and a control button used to perform preparation work before image capture, such as patient selection, protocol selection, positioning, etc.

The wall stand apparatus 120 includes a first detector assembly 121, a wall stand 122, and a connecting portion 123. The connecting portion 123 includes a support arm that is vertically connected in the height direction of the wall stand 122 and a rotating bracket that is mounted on the support arm, and the first detector assembly 121 is mounted on the rotating bracket. The wall stand apparatus 120 further includes a detector driving apparatus that is arranged between the rotating bracket and the first detector assembly 121, which is driven by the detector driving apparatus to move in a direction parallel to the height direction of the wall stand 122 in the plane held by the rotating bracket, and the first detector assembly 121 can further be rotated relative to the support arm to form an angle with the wall stand. The first detector assembly 121 has a plate-like structure, the orientation of which is variable, facilitating an X-ray incident surface to become vertical or horizontal depending on the incident direction of the X-rays.

A second detector assembly 131 is included on an examination table apparatus 130, and the selection or use of the first detector assembly 121 and the second detector assembly 131 may be determined on the basis of an image capture site of a patient and/or an image capture protocol, or may be determined on the basis of the location of the subject under examination obtained by image capture of a camera, so as to perform image capture and examination in a lying or standing position. FIG. 1 only shows an example diagram of a wall stand and an examination table, and it should be understood by those skilled in the art that wall stands and/or examination tables of any form or arrangement can be selected, or only the wall stand can be mounted, and the wall stand and/or examination table is not intended to limit the overall solution of the present application.

The X-ray imaging system further includes a control apparatus (not shown in the figures). The control apparatus may be a main controller located in a control room, a tube console mounted on the suspension apparatus, a mobile or portable controller, or any combination of the above. The control apparatus may include a source controller and a detector controller. The source controller is used to command an X-ray source to emit X-rays for image exposure. The detector controller is used to select a suitable detector from among a plurality of detectors and to coordinate the control of various detector functions, such as automatically selecting a corresponding detector according to the position or pose of the subject under examination. Alternatively, the detector controller may perform various signal processing and filtering functions, specifically, for initial adjustment of a dynamic range, interleaving of digital image data, and the like. In some embodiments, the control apparatus may provide power and timing signals for controlling the operation of the X-ray source and the detector.

In some embodiments, the control apparatus may further be configured to use a digitized signal to reconstruct one or more required images and/or determine useful diagnostic information corresponding to the patient, wherein the control apparatus may include one or more dedicated processors, graphics processing units, digital signal processors, microcomputers, microcontrollers, application-specific integrated circuits (ASICs), field-programmable gate arrays (FPGAs), or other appropriate processing apparatuses.

Certainly, the X-ray imaging system may further include other numbers or configurations or forms of control apparatuses, for example, the control apparatus may be local (e.g., co-located with one or more X-ray imaging systems 100, e.g., within the same facility and/or the same local network). In other implementations, the control apparatus may be remote, and thus only accessible by means of a remote connection (for example, by means of the Internet or other available remote access technologies). In a specific implementation, the control apparatus may also be configured in a cloud-like means, and may be accessed and/or used in a means that is substantially similar to the means by which other cloud-based systems are accessed and used.

In one embodiment, the X-ray imaging system 100 further includes an operator workstation, the operator workstation allowing the user to receive and evaluate the reconstructed image, and input a control instruction (an operation signal or a control signal). The operator workstation may include a user interface (or a user input device) having a certain form of operator interface, such as a keyboard, a mouse, a voice activated controller, or any other suitable input device, and an operator may input an operation signal/control signal to the control apparatus by means of the user interface.

The movement of the suspension apparatus 110 includes the movement of the tube assembly along the x-axis, y-axis, and z-axis, as well as the rotation of the tube assembly in the horizontal plane (the axis of rotation is parallel to or overlaps with the z-axis) and in the vertical plane (the axis of rotation is parallel to the y-axis). In the above motion, a motor is usually used to drive a rotating shaft which in turn drives corresponding components to rotate in order to achieve the corresponding movement or rotation, and the corresponding control components are generally mounted in the sliding member 114. An X-ray imaging unit further includes a motion control unit (not shown in the figure), and the motion control unit can control the described motion of the suspension apparatus 110. Furthermore, the motion control unit can receive a control signal to control a corresponding component to carry out motion correspondingly.

Specifically, the suspension apparatus includes a driving assembly 300 disposed within the sliding member 114, the suspension apparatus further includes a frame structure 310, and the frame structure 310 is used to mount the driving assembly 300. Specifically, the frame structure 310 is formed by bending a processed plate material, the frame structure 310 has a hollow portion, and the frame structure 310 includes a reinforcing member capable of dividing the hollow portion into a first hollow portion and a second hollow portion.

The driving assembly 300 may include a rotating shaft 301, a drum 302, a motor 303, synchronous wheels and synchronous belts, and other components, which are disposed in the first hollow portion. The motor 303 can drive the drum 302 to rotate about the rotating shaft 301 by means of the synchronous wheels and the synchronous belts, so as to drive the telescopic cylinder 113 to move along the z-axis. The driving assembly 300 may include a balancing apparatus 305 disposed in the second hollow portion and used for balancing the telescopic cylinder and/or the tube assembly.

In some embodiments, the driving assembly 300 further includes wire ropes, wherein the wire ropes include a main wire rope having one end fixed to the drum 302 and the other end fixed to the top of the lowermost cylinder shell of the telescopic cylinder 113, an auxiliary wire rope having one end fixed to the drum 302 and the other end fixed to the top of the lowermost cylinder shell of the telescopic cylinder, and a connecting wire rope having one end fixed to the balancing apparatus 305 and the other end fixed to the drum 302. The motor 303 can drive the drum 302 to rotate so as to wind or release wire ropes (including the main wire rope and the auxiliary wire rope), thereby driving the telescopic cylinder 113 to raise or lower.

A guide rail driving assembly 330 is provided at the intersection between the transverse guide rail 112 and the longitudinal guide rail 111. The guide rail driving assembly 330 is mounted at the bottom of the longitudinal guide rail 111 and at a side edge of the transverse guide rail 112. The guide rail driving assembly 330 can drive the transverse guide rail 112 to move relative to the longitudinal guide rail 111, thereby driving the telescopic cylinder 113 and the tube assembly 115 to move along the x-axis direction.

Further included within the sliding member 114 is a short-axis driving assembly 340 mounted outside the frame structure 310. The short-axis driving assembly 340 can drive the sliding member 114 to move relative to the transverse guide rail 112, i.e., moving along the y-axis direction. The short-axis driving assembly 340 includes components such as a motor, a synchronous belt, and an encoder.

Since the driving assembly for controlling the movement of the suspension apparatus is basically arranged inside the sliding member, when a certain part or component of the driving assembly fails, the suspension apparatus will immediately stop moving and remain in its current position, which requires a field engineer to check and repair in this case. In particular, during the repair process, for example, when cables, the balancing apparatus, or the components in the tube assembly need to be replaced, the telescopic cylinder or the tube assembly or the balancing apparatus needs to be removed, and the replacement is then installed onto the suspension apparatus. However, components such as the telescopic cylinder and the tube assembly are all heavy, so that a hoisting mechanism is required for removal, replacement, and reinstallation of the respective components. The entire process requires the acquisition of the hoisting mechanism, and the cost and time required are unpredictable.

Furthermore, the field engineer needs to check the cause of the failure, order the required parts, and replace them only after new replacement parts are acquired. The time required for this process is unpredictable, and the height or position of conventional suspension apparatuses cannot be adjusted after a malfunction, so that the existing image capture task cannot be completed, thereby affecting the image capture progress for the hospital.

In view of the above problems, the present application proposes a spare assembly capable of being mounted adjacent to the rotating shaft of the driving assembly. The spare assembly includes a worm wheel mounted on the rotating shaft and capable of rotating along with the rotating shaft, and a worm mounted on a support of the driving assembly and disposed at a certain distance from the worm wheel. When the driving assembly in normal operation, there is a certain distance between the worm wheel and the worm, and they do not interfere with each other, so that the worm wheel can rotate along with the rotating shaft. When the driving assembly fails, the worm can be adjusted to move toward the worm wheel by a predetermined distance, so that the worm wheel and the worm are engaged with each other. Then, the worm can be rotated to drive the worm wheel to rotate through engagement, thereby driving the rotating shaft to rotate, and accordingly driving the telescopic cylinder and other components to move. Therefore, by mounting the worm wheel and worm assembly on the driving assembly, when the driving assembly fails, the telescopic cylinder, the balancing apparatus, and other components can be lowered in height without requiring a hoisting mechanism for maintenance and replacement. Moreover, during the wait for maintenance, the suspension apparatus can be adjusted to an appropriate height, so that some simple image capture operations can be performed. In addition, the entire worm wheel and worm assembly has a compact size with little space being occupied, is low cost, and facilitates maintenance.

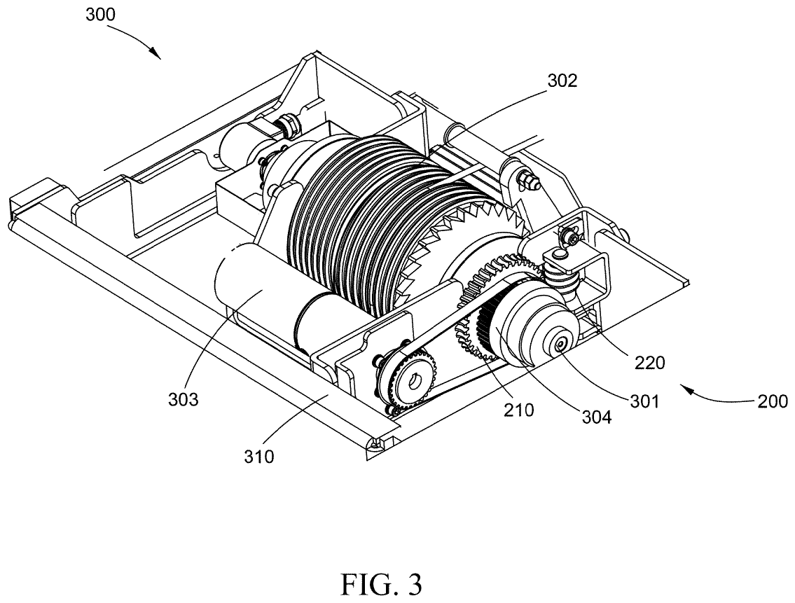

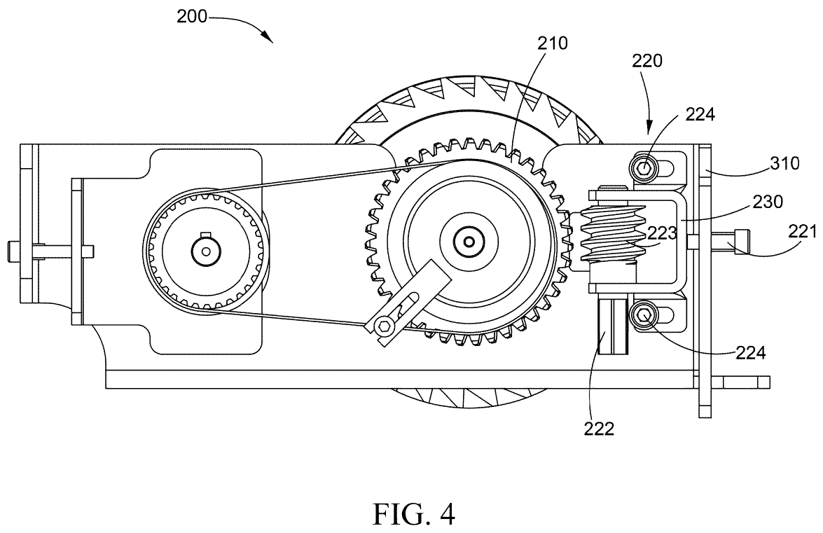

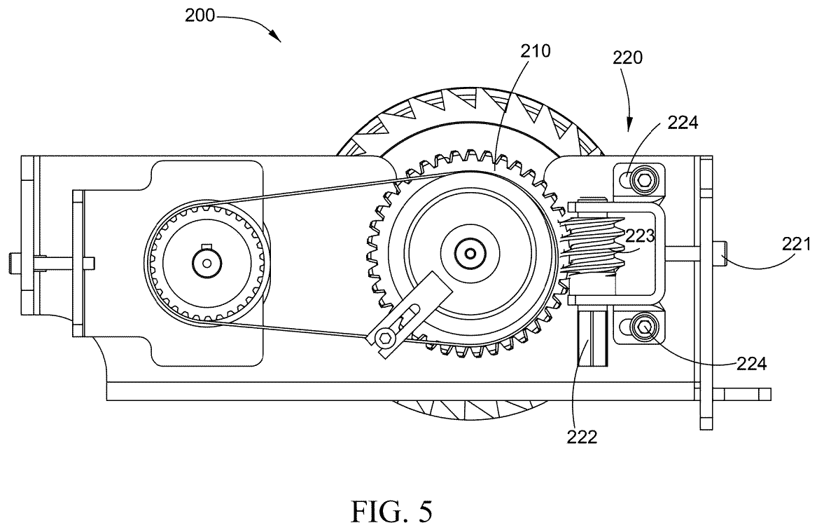

FIG. 3 shows a schematic diagram of a spare assembly 200 according to some embodiments of the present invention, FIG. 4 shows a schematic diagram of the spare assembly 200 in a first state according to some embodiments of the present invention, and FIG. 5 shows a schematic diagram of the spare assembly 200 in a second state according to some embodiments of the present invention. As shown in FIGS. 3 to 5, the spare assembly 200 includes a worm wheel portion 210 and a worm portion 220, the worm wheel portion 210 is mounted on the rotating shaft 301, the worm portion 220 is mounted at a predetermined distance from the worm wheel portion 210, and the worm portion 220 has a first adjusting portion 221 and a second adjusting portion 222. The first adjusting portion 221 is operable to engage the worm portion 220 with the worm wheel portion 210, and the second adjusting portion 222 is operable to drive the worm wheel portion 210 by means of the worm portion 220, so as to drive the rotating shaft 301 to move.

In some embodiments, the worm wheel portion 210 is mounted adjacent to the rotating shaft 301. Specifically, the worm wheel portion 210 is mounted at a position closest to the drum 302. For example, the worm wheel portion is mounted closer to the drum 302 than the synchronous wheel 304 and a clutch are. Such an installation manner reduces the offset of the worm wheel portion.

In some embodiments, the worm portion 220 is mounted on a side edge of the worm wheel portion 210, and the second adjusting portion 222 is located at a bottom portion of the worm portion 220. Specifically, the worm portion 220 may be mounted at an arbitrary side of the worm wheel portion 210, for example, at the bottom, top, or the left or right side of the worm wheel portion. The position of the second adjusting portion 222 may be determined depending on whether the operator's operation is facilitated. For example, when the worm portion 220 is mounted at the left or right side of the worm wheel portion 210, the second adjusting portion can be mounted at the bottom of the worm portion to facilitate the operator's control of the worm wheel portion by means of the second adjusting portion.

In some embodiments, the worm portion 220 further has a body 223, and the second adjusting portion 222 is mounted at the bottom of the body 223, or the second adjusting portion 222 can pass through a portion of the body 223 so as to be fixed, and the second adjusting portion 222 is operable to enable the body 223 to rotate. The body 223 is provided with a screw thread which may be engaged with the worm wheel portion 210, that is, the second adjusting portion 222 is operable to enable the screw thread to rotate.

The worm portion 220 is fixed to the frame structure 310 by means of a worm frame 230. In some embodiments, the worm frame 230 is substantially U-shaped, and includes a side wall and top and bottom surfaces respectively connected at two ends of the side wall, with the body 223 being fixed between the bottom and top surfaces of the U-shaped worm frame 230. The second adjusting portion 222 is fixed to the bottom surface of the U-shaped worm frame 230.

The first adjusting portion 221 can abut against the side wall of the U-shaped worm frame 230 through the frame structure 310, that is, one end of the first adjusting portion 221 is in contact with the outer side wall of the worm frame 230. Specifically, the first adjusting portion 221 is operable to move the worm frame 230 toward the worm wheel portion 210, so as to drive the body 223 and the second adjusting portion 222 to move toward the worm wheel portion 210, thereby enabling the worm wheel portion 210 to engage with the worm portion 220. Specifically, the first adjusting portion 221 may be a screw, and the distance between the screw and the frame structure 310 is substantially equal to the distance between the body 223 of the worm portion 220 and the worm wheel portion 210.

In some embodiments, the worm portion 220 further includes a limiting apparatus 224, which may be provided at the worm frame 230, and can limit the maximum distance of movement of the worm frame 230, i.e., the limit position of the worm frame 230. The limiting apparatus 224 includes a limiting slot and a screw. At the initial position, the first adjusting portion 221 is at a predetermined distance from the frame structure 310, and the screw is located at a first position in the limiting slot. At this time, the worm wheel portion 210 is also at the predetermined distance from the worm portion 220. When the first adjusting portion 221 is operated to move the worm portion 220 towards the worm wheel portion 210, and when the screw is located at a second position in the limiting slot, the worm wheel portion 210 can exactly engage with the worm portion 220. Specifically, the worm portion 220 includes two limiting apparatuses, which are mounted on the top and bottom surfaces of the worm frame 230, respectively.

Specifically, the second adjusting portion can control the movement of the rotating shaft when the driving assembly fails, thereby driving an assembly connected to the rotating shaft to move.

In some embodiments, the spare assembly 200 has a first state and a second state. In the first state, the worm wheel portion 210 and the worm portion 220 have a predetermined distance therebetween and do not interfere with each other, and the worm wheel portion 210 can rotate along with the rotating shaft 301 and the drum 302, etc. In the second state, a failure occurs in the driving assembly 300 of the suspension apparatus or some component(s) in the telescopic cylinder 113, and the suspension apparatus stops moving. The worm portion 220 can be moved (by adjusting the first adjusting portion 221) to engage with the worm wheel portion 210, and the second adjusting portion 222 of the worm portion 220 can be adjusted to rotate the worm wheel portion 210, so as to drive the rotating shaft 301 and the drum 302 to rotate, thereby driving the driving assembly 300 to move.

In some embodiments, the adjustment or control of the second adjusting portion 222 may be manually implemented using components such as a wrench or a screw, or may be implemented using devices such as an electric wrench or an electric instrument. In some embodiments, the adjustment or control of the first adjusting portion 221 may be manually implemented by an operator, or may be automatically controlled by means of a control signal from a controller. For example, when a failure occurs in the driving assembly, the controller can send a control signal to the first adjusting portion 221, so as to control the movement of the first adjusting portion 221 to achieve engagement.

In some embodiments, the spare assembly 200 can be used to drive the rotating shaft 301, so as to drive the telescopic cylinder 113 to raise and lower. In some other embodiments, the spare assembly 200 can be used to drive the rotating shaft 301, so as to drive the balancing apparatus to raise or lower. The two embodiments will be described separately below.

First, in some embodiments, the second adjusting portion of the worm portion is operable to drive the telescopic cylinder to raise or lower. Specifically, when it is necessary to raise or lower the telescopic cylinder in the event of failure of the driving assembly, the operation can be performed by means of the following steps: first, the worm portion and the worm wheel portion can be engaged by adjusting the first adjusting portion of the worm portion. Next, by adjusting the second adjusting portion, the telescopic cylinder reaches the topmost position at which all cylinder shells of the telescopic cylinder are collapsed together. Each cylinder shell is provided with a fixing hole, and by inserting a fixing pin into the cylinder shells, the relative positions between the plurality of cylinder shells can be fixed, that is, the telescopic cylinder can no longer be extended or collapsed, thus facilitating the subsequent raising or lowering and replacement. Then, a screw or other fixing members between the top of the telescopic cylinder and the sliding member is unfastened, and in this case, the entire weight of the telescopic cylinder is borne by the main wire rope and the auxiliary wire rope. Further, the second adjusting portion of the worm portion is driven so that the entire telescopic cylinder can be lowered, and then replacement of parts is performed. Finally, the second adjusting portion is adjusted so that the telescopic cylinder after the maintenance can be raised to the topmost position, the telescopic cylinder is re-fixed to the sliding member, and the fixing pin is pulled out, thereby completing the entire maintenance and replacement work for the telescopic cylinder. In this process, the telescopic cylinder can be raised or lowered without a hoisting mechanism.

In some other embodiments, when the driving assembly fails but a new component or assembly has not yet arrived, in this case, by directly adjusting the second adjusting portion, the telescopic cylinder can be adjusted to an appropriate height to perform some simple image capture operations, so that some image capture operations can still continue during the wait for maintenance, preventing the image capture workflow from being completely disrupted.

In still other embodiments, when the tube assembly needs to be replaced, control work may also be performed by the spare assembly, which is specifically as follows: first, the telescopic cylinder is moved to an appropriate height by the second adjusting portion. Then, the tube assembly is detached from the telescopic cylinder. Next, when a new tube assembly needs to be remounted, said tube assembly can be placed in a fixed position, and then the position of the telescopic cylinder can be adjusted by adjusting the second adjusting portion so that the telescopic cylinder is aligned with the tube assembly, thereby completing the assembly. Such a process eliminates the need to align the tube assembly with the telescopic cylinder, which can save manpower and material resources required to adjust the position of the tube assembly.

Furthermore, the second adjusting portion of the worm portion can be further operated to drive the balancing apparatus to raise or lower. Specifically, when it is necessary to raise or lower the telescopic cylinder in the event of failure of the driving assembly, the operation can be performed by means of the following steps: first, the worm portion and the worm wheel portion can be engaged by adjusting the first adjusting portion of the worm portion. Next, by adjusting the second adjusting portion, the telescopic cylinder reaches the topmost position, and the fixing pin is inserted into the cylinder shells. Then, the wire ropes (including the main wire rope and the auxiliary wire rope) between the drum and the telescopic cylinder are unfastened. The wire ropes can be fixed at a certain position or can be completely unfastened. Next, the auxiliary wire rope or another completely new wire rope is fixed between the drum and the balancing apparatus; alternatively, the original wire rope between the drum and the balancing apparatus is directly used, with one end of the wire rope being fixed to the drum and the other end being fixed to the balancing apparatus by means of a hanging point on a support of the balancing apparatus, which will be described in detail below. Next, a screw or other fixing elements between the balancing apparatus and the frame structure or the sliding member are unfastened. Further, the second adjusting portion of the worm portion is driven so that the entire balancing apparatus can be lowered, and then replacement of parts is performed. Finally, the second adjusting portion is adjusted so that the new balancing apparatus can be raised to the topmost position, the balancing apparatus is re-fixed to the frame structure or the sliding member, and the wire ropes between the telescopic cylinder and the drum are replaced or mounted, thereby completing the entire maintenance and replacement work for the balancing apparatus. In this process, the balancing apparatus can be raised or lowered without a hoisting tool.

In addition to being applied to the driving assembly of a suspension apparatus, the spare assembly proposed in the present application can also be applied to driving assemblies of other types of X-ray imaging systems, and surely can also be applied to other types of medical devices, including but not limited to computerized tomography (CT) systems, positron emission tomography (PET) systems, magnetic resonance imaging (MRI) systems, etc.

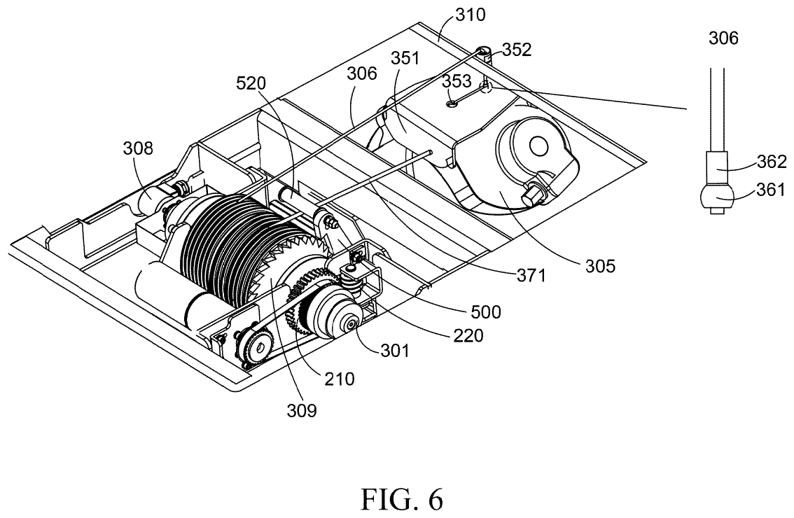

FIG. 6 shows a schematic diagram of a driving assembly according to some embodiments of the present application. As shown in FIG. 6, the balancing apparatus 305 includes a mounting support 351, and the balancing apparatus 305 can be fixed onto the frame structure 310 by means of the mounting support 351. Specifically, the mounting support 351 is located at the top of the balancing apparatus 305. In order to facilitate raising or lowering of the balancing apparatus by using the spare assembly, a hanging point 352 and a wire rope fixing portion 353 are further provided on the balancing apparatus 305 or the mounting support 351.

Specifically, the hanging point 352 may be provided on the frame structure 310 or the mounting support 351 as long as the hanging point 352 is positioned in the vertical direction of the center of gravity of the balancing apparatus. The wire rope fixing portion 353 is mounted on the top of the mounting support 351, and a spare wire rope 306 can be fixed to the wire rope fixing portion 353 by means of the hanging point 352, so as to control raising or lowering of the balancing apparatus by means of the second adjusting portion. Specifically, the spare wire rope 306 may be the main wire rope or the auxiliary wire rope or another new wire rope, and the spare wire rope is only used when the balancing apparatus needs to be raised or lowered during maintenance.

Specifically, by providing the hanging point 352, the spare wire rope 306 can be connected to a position relating to the center of gravity of the balancing apparatus 305 when the balancing apparatus 305 is being raised or lowered, so that the balancing apparatus does not shake during the raising or lowering process.

The wire rope fixing portion 353 has a slot and a through-hole provided at one end of the slot. The through-hole is in communication with the slot, the diameter of the through-hole is slightly larger than the width of the slot, and the width of the through-hole is determined according to the size of an end portion of the spare wire rope. Specifically, the end portion of the spare wire rope 306 has a first protrusion 361 and a second protrusion 362. For example, the first protrusion 361 may be a sphere, and the second protrusion 362 may be a cylinder. The first protrusion 361 is closer to the end portion of the spare wire rope than the second protrusion 362, and the first protrusion 361 has a larger diameter than that of the second protrusion 362. Moreover, the width of the slot of the wire rope fixing portion 353 is smaller than the diameter of the first protrusion 361 and larger than the diameter of the second protrusion 362, and the width of the through-hole of the wire rope fixing portion 353 is larger than the diameter of the first protrusion 361. Such a configuration enables the entire first protrusion 361 and the entire second protrusion 362 of the spare wire rope 306 to be placed into the through-hole, and then the wire rope can be moved into the slot. Since the diameter of the first protrusion 361 is larger than the width of the slot, the first protrusion 361 is stuck at the bottom of the slot, so that the wire rope can be fixed.

Generally, the end of the slot away from the through-hole in the wire rope fixing portion 353 is located in the same vertical direction as the hanging point 352.

Therefore, by providing the hanging point and the wire rope fixing portion on the balancing apparatus, the mounting support of the balancing apparatus, or the frame structure, the wire rope can be fixed at the position relating to the center of gravity of the balancing apparatus so that the balancing apparatus does not significantly shake during the raising or lowering process, and the wire rope can be fixed simply by means of structural design without using other fixing means such as a screw.

The above embodiment describes the solution in which the main wire rope and the auxiliary wire rope are separately connected between the drum and the telescopic cylinder, and the connecting wire rope is provided between the drum and the balancing apparatus. In order to further save costs, the driving assembly may also use one less wire rope by adopting the solution of wire rope direct connection. Specifically, one end of the main wire rope may be fixed to the top of the lowermost cylinder shell of the telescopic cylinder, and the other end may be wound around the drum and then connected onto the balancing apparatus, and one end of the auxiliary wire rope may be fixed onto the drum, and the other end may be fixed to the top of the lowermost cylinder shell of the telescopic cylinder.

In some embodiments, the main wire rope and the auxiliary wire rope can be wound around the drum in an alternating manner, or the main wire rope is wound around one end of the drum and the auxiliary wire rope is wound around the other end of the drum. Specifically, the main wire rope may be wound at the end close to the worm wheel portion, and the auxiliary wire rope may be wound at the other end of the drum. Of course, the directions may also be interchanged, and the present application does not impose any restrictions in this regard.

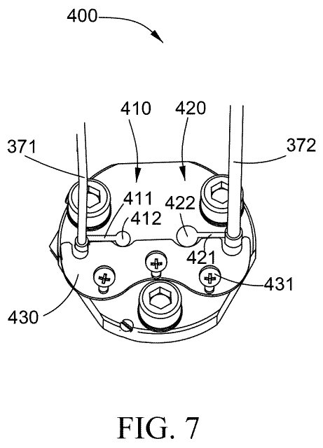

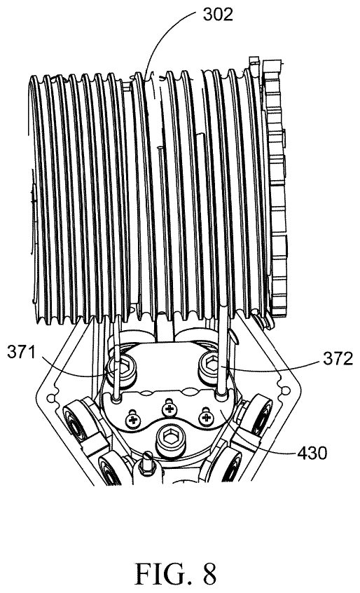

FIG. 7 shows a schematic diagram of a wire rope fixing support 400 in a first position according to some embodiments of the present application, and FIG. 8 shows a schematic diagram of the wire rope fixing support 400 in a second position according to some embodiments of the present application. As shown in FIGS. 6 to 8, the driving assembly includes a main wire rope 371 and an auxiliary wire rope 372, where one end of the main wire rope 371 is fixed to the lowermost cylinder shell of the telescopic cylinder by means of the wire rope fixing support 400 and the other end is fixed inside the balancing apparatus by means of the drum 302 (the wire rope 371 as shown in FIG. 6), and one end of the auxiliary wire rope 372 is fixed onto the wire rope fixing support 400 and the other end is fixed onto the drum 302.

In some embodiments, a mounting groove is provided on a side surface of the drum 302 around which an end of the auxiliary wire rope 372 is wound, and a spring is mounted on the end of the auxiliary wire rope 372. The end of the auxiliary wire rope 372 having the spring is fixed inside the mounting groove of the drum. By providing the spring, the auxiliary wire rope can bear weight without being in a tensioned state, and when the main wire rope is broken, the auxiliary wire rope can thus bear weight. Specifically, the auxiliary wire rope having the spring may be fixed inside the mounting groove in any suitable manner, for example, in the form of a hook, a screw, etc.

Specifically, the wire rope fixing support 400 is fixed to the inside of the lowermost cylinder shell. Specifically, it may be fixed to the inside near the top of the lowermost cylinder shell, and the wire rope fixing support 400 has a substantially circular or hexagonal cross-section. Surely, the wire rope fixing support may also be configured to have other shapes as long as the shape matches the cross-sectional shape of the cylinder shell.

In some embodiments, the wire rope fixing support 400 includes a body portion and a cover plate. The body portion includes at least one opening for receiving the wire ropes. The cover plate is movable relative to the body portion to cover at least a portion of the opening to fix the positions of the wire ropes.

Similar to the structure of the end portion of the wire rope shown in FIG. 6, the end portions of the main wire rope and the auxiliary wire rope connected to the telescopic cylinder each have a first protrusion and a second protrusion.

Specifically, a main wire rope fixing portion 410 and an auxiliary wire rope fixing portion 420 are provided on the body portion. Similar to the structure of the wire rope fixing portion 353 provided on the balancing apparatus shown in FIG. 6, the main wire rope fixing portion 410 and the auxiliary wire rope fixing portion 420 each include a slot and a through-hole provided at one end of the slot. Specifically, the main wire rope fixing portion 410 includes a first slot 411 and a first through-hole 412, and the auxiliary wire rope fixing portion 420 includes a second slot 421 and a second through-hole 422, wherein the first through-hole 412 and the second through-hole 422 are provided adjacent to each other. The first protrusion and the second protrusion provided at the end portion of the main wire rope 371 can enter the first through-hole 412 and then slide into the other end of the first slot 411. Since the width of the first slot 411 is smaller than the diameter of the first protrusion of the main wire rope and larger than the diameter of the second protrusion, the main wire rope fixing portion 410 can fix the main wire rope 371 in the vertical direction. Similarly, the protrusions provided at the end portion of the auxiliary wire rope 372 can enter the second through-hole 422 and then slide into the other end of the second slot 421, thereby fixing the auxiliary wire rope in the vertical direction.

Further, in order to fix the main wire rope 371 and the auxiliary wire rope 372 in the horizontal direction, a cover plate 430 is further added to the body provided with the main wire rope fixing portion 410 and the auxiliary wire rope fixing portion 420. The cover plate 430 is located on the side of the main wire rope fixing portion 410 and the auxiliary wire rope fixing portion 420. The cover plate 430 can be moved to fix the main wire rope 371 and the auxiliary wire rope 372 in the horizontal direction so that the wire ropes cannot slide relative to the slots.

Specifically, the cover plate 430 is generally in the shape of a long strip, and one side of the cover plate 430 has two recesses each adjacent to an end position, and the size of the recesses is determined by the thickness (i.e., the diameter) of the wire ropes. For convenience of description, the end of the first slot 411 away from the first through-hole 412 and the end of the second slot 421 away from the second through-hole 422 are defined as the end positions.

In some embodiments, the cover plate 430 has a first position and a second position. In the first position, the first slot 411, the first through-hole 412, the second slot 421, and the second through-hole 422 can all be displayed to facilitate installation and replacement of the wire ropes by an operator. In the second position, the cover plate 430 is moved to cover most of the first slot 411, the first through-hole 412, the second slot 421, and the second through-hole 422, leaving only the wire ropes in the recesses, and thereby fixing the wire ropes in the horizontal direction.

In some embodiments, the cover plate 430 further includes at least one limiting strip 431. The limiting strip 431 can define the maximum distance that the cover plate 430 can move, that is, the limiting strip can define the first position and the second position at which the cover plate 430 is located. Specifically, the limiting strip 431 includes a limiting slot and a screw, the position of the screw is fixed, and the cover plate can move from one end to the other end of the limiting slot, that is, the cover plate 430 is switched from the first position to the second position or from the second position to the first position.

With further reference to FIG. 6, the driving assembly further includes a position feedback unit for providing position feedback regarding movement in the vertical direction. In some embodiments, the position feedback unit may include an encoder 308, and the encoder 308 may be mounted at one end of the rotating shaft 301 to provide position feedback by monitoring the rotation of the rotating shaft.

In other embodiments, the position feedback unit may include a line encoder (not shown). The line encoder may be mounted at any position on the frame structure, for example, the position on the frame structure close to the motor, or the position on the frame structure between the drum and the motor. The line encoder includes a connecting line, one end of which is fixed inside the line encoder and the other end of which may be mounted on the lowermost cylinder shell of the telescopic cylinder, for example, on the wire rope fixing support as shown in FIG. 7. That is, the connecting line can be pulled out or displaced along with the vertical movement of the telescopic cylinder, so that the line encoder can provide feedback on the vertical movement distance on the basis of the distance by which the connecting line is pulled out or displaced.

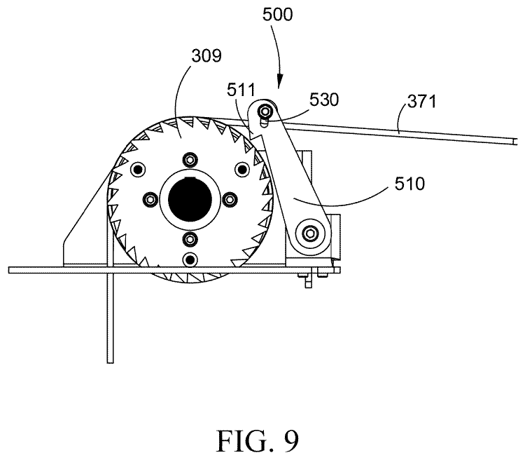

FIG. 9 shows a cross-sectional view of a safety locking mechanism according to some embodiments of the present application. As shown in FIGS. 6 and 9, in some embodiments, the driving assembly 300 further includes a ratchet 309 mounted on an end of the drum 302. In some embodiments, the ratchet 309 is positioned closer to the drum than the worm wheel portion 210 is. Specifically, the ratchet 309, the worm wheel portion 210, the synchronous wheel, the clutch, and other components may be arranged in a direction from closer to the drum to away from the drum.

The driving assembly 300 further includes a safety locking mechanism 500. The safety locking mechanism 500 is mounted adjacent to the ratchet 309, and the safety locking mechanism 500 can engage with the ratchet 309 to lock the rotating shaft 301 when the main wire rope 371 is broken.

Specifically, the safety locking mechanism 500 includes a rotating portion 510 and an extension portion 520, the extension portion 520 is connected to the rotating portion 510, the end portion of the rotating portion 510 is provided with a gear 511, and the gear 511 can engage with the ratchet 309.

Specifically, the rotating portion 510 is fixed onto the frame structure 310, and can rotate relative to the frame structure 310, and the rotating portion 510 can be oriented toward the ratchet 309 and be positioned. Specifically, the rotating portion 510 may further be fixed onto the frame structure 310 by means of a spring.

The extension portion 520 is positioned along the length direction of the rotating shaft 301, the plane in which the extension portion 520 is located is perpendicular to the plane in which the rotating portion 510 is located, and the extension portion 520 can be fixed on the wire rope between the drum and the balancing apparatus. Moreover, the extension portion 520 can be in contact with the wire rope, and the wire rope can provide a certain support for the extension portion 520. By bringing the extension portion 520 into contact with the wire rope, the rotating portion 510 is positioned at a certain distance from the ratchet 309, so that the rotating portion 510 and the ratchet 309 do not interfere with each other. When the wire rope is broken or other failures occur, the extension portion 520 loses its support, and the rotating portion 510 also loses its support due to the spring or gravity, and rotates toward the ratchet 309, so that the gear 511 provided at the end portion of the rotating portion 510 is engaged with the ratchet 309, thereby locking the rotating shaft 301.

The safety locking mechanism 500 further includes an adjusting unit 530, which may be provided at a connection location between the rotating portion 510 and the extension portion 520, and the adjusting unit 530 can be used to adjust a preset distance between the gear 511 and the ratchet 309. Specifically, the adjusting unit 530 includes an adjusting slot and a screw, and the relative distance between the gear and the ratchet can be adjusted by adjusting the position of the screw in the adjusting slot. By providing the adjusting unit, it is possible to minimize the distance of the gear 511 from the ratchet 309 without hindering the rotation of the ratchet, so that the triggering distance of the safety locking mechanism is minimized, that is, when the main wire rope is broken, the safety locking mechanism can be triggered in the shortest time to lock the rotating shaft.

Accordingly, by arranging, in the vicinity of the ratchet, the rotating portion provided with the gear and the extension portion supported by the wire rope, it is possible to self-lock the rotating shaft when the wire rope is broken, thus improving the safety of the entire suspension apparatus.

The spare assembly according to some embodiments of the present invention includes a worm wheel and a worm, and the worm wheel can rotate along with a rotating shaft. When the driving assembly fails, the worm can be moved to engage with the worm wheel, so as to lock the rotating shaft and prevent displacement. Further, a second adjusting portion is provided on the worm, and an operator can control rotation of the rotating shaft by means of the second adjusting portion, so as to raise or lower the telescopic cylinder and the balancing apparatus, thereby eliminating the time and cost involved in raising and lowering a hoisting tool. Furthermore, in the event of a failure in the driving assembly, during the wait for new replacement parts, the operator can adjust the telescopic cylinder to an appropriate height by operating the second adjusting portion, so that the suspension apparatus can still perform some simple image capture operations, meeting certain image capture requirements.

Furthermore, some improvements are further made to the driving assembly in the present application. The main wire rope is directly fixed between the telescopic cylinder and the balancing apparatus by means of wire rope direct connection, which saves the cost of one wire rope. Furthermore, the wire rope fixing support is mounted inside the lowermost cylinder shell of the telescopic cylinder, and can be used to fix the wire ropes. For example, by using the shape design of the end portions of the wire ropes, and by configuring the shape of the opening, the wire ropes can be fixed in the vertical direction, and then by moving the position of the cover plate, the wire ropes can be caught in the recesses, thereby fixing the wire ropes in the horizontal direction. The structure is simple, and the cost is low. Finally, the safety locking mechanism is provided on the driving assembly, which can lock the rotating shaft and prevent displacement, etc., when the wire rope is broken.

The exemplary embodiments of the present application provide a spare assembly for a medical imaging system. The medical imaging system comprises a driving assembly, the driving assembly comprising a rotating shaft, and the spare assembly comprising: a worm wheel portion mounted on the rotating shaft; and a worm portion mounted at a predetermined distance from the worm wheel portion, the worm portion having a first adjusting portion and a second adjusting portion, the first adjusting portion being operable to engage the worm portion with the worm wheel portion, and the second adjusting portion being operable to drive the worm wheel portion by means of the worm portion, so as to drive the rotating shaft to move.

Specifically, the second adjusting portion is capable of controlling movement of the rotating shaft when the driving assembly fails, thereby driving an assembly connected to the rotating shaft to move.

Specifically, the worm portion is mounted at a side edge of the worm wheel portion, and the second adjusting portion is located at a bottom portion of the worm portion.

Specifically, the medical imaging system includes a suspension apparatus, the suspension apparatus includes a set of perpendicularly mounted guide rails, a sliding member, and a telescopic cylinder, the guide rails are mounted on a ceiling, the sliding member is mounted on the guide rails, the sliding member is connected to the telescopic cylinder, the driving assembly is mounted within the sliding member to drive the telescopic cylinder to move, and the second adjusting portion of the worm portion is operable to drive the telescopic cylinder to raise or lower.

Specifically, the driving assembly further includes a balancing apparatus, and the second adjusting portion of the worm portion is further operable to drive the balancing apparatus to raise or lower.

Specifically, a top portion of the balancing apparatus is provided with a wire rope fixing portion and a hanging point, the handing point is provided at a position relating to the center of gravity of the balancing apparatus, and a wire rope can be fixed to the wire rope fixing portion by means of the hanging point, so as to control raising or lowering of the balancing apparatus by means of the second adjusting portion.

The exemplary embodiments of the present application provide a suspension apparatus. The suspension apparatus comprises a set of perpendicularly mounted guide rails, a sliding member, and a telescopic cylinder, the guide rails being mounted on a ceiling, the sliding member being mounted on the guide rails, the sliding member being connected to the telescopic cylinder, and the driving assembly being mounted within the sliding member to drive the telescopic cylinder to move. The driving assembly comprises a rotating shaft, and the spare assembly comprises: a worm wheel portion mounted on the rotating shaft; and a worm portion mounted at a predetermined distance from the worm wheel portion, the worm portion having a first adjusting portion and a second adjusting portion, the first adjusting portion being operable to engage the worm portion with the worm wheel portion, and the second adjusting portion being operable to drive the worm wheel portion by means of the worm portion, so as to drive the rotating shaft to move.

Specifically, the driving assembly further includes: a drum rotatable about the rotating shaft; a main wire rope, one end of which is fixed onto the balancing apparatus and the other end of which is fixed onto the telescopic cylinder by means of the drum; and an auxiliary wire rope, one end of which is fixed onto the drum and the other end of which is fixed onto the telescopic cylinder.

Specifically, the main wire rope and the auxiliary wire rope are capable of being wound around the drum in an alternating manner, or the main wire rope is wound around one end of the drum and the auxiliary wire rope is wound around the other end of the drum.

Specifically, the telescopic cylinder includes a plurality of cylinder shells which are sleeved from top to bottom, where a wire rope fixing support is mounted at a top portion of the lowermost cylinder shell, the wire rope fixing support includes a body portion and a cover plate, the body portion includes at least one opening for receiving the wire ropes, the cover plate is movable relative to the body portion to cover at least a portion of the opening to fix the positions of the wire ropes.

Specifically, the driving assembly further includes a line encoder mounted adjacent to the drum, the line encoder including a connecting line, and the other end of the connecting line being fixed to the lowermost cylinder shell.

Specifically, one end of the drum is provided with a ratchet, and the driving assembly further comprises a safety locking mechanism, the safety locking mechanism being mounted adjacent to the ratchet, and being capable of engaging with the ratchet to lock the rotating shaft when the main wire rope is broken.

Specifically, the safety locking mechanism includes a rotating portion and an extension portion, the extension portion is connected to the rotating portion, an end portion of the rotating portion is provided with a gear, and the gear is capable of engaging with the ratchet.

The exemplary embodiments of the present application provide an X-ray imaging system. The X-ray imaging system comprises a suspension apparatus, and the suspension apparatus comprises a set of perpendicularly mounted guide rails, a sliding member, and a telescopic cylinder, the guide rails being mounted on a ceiling, the sliding member being mounted on the guide rails, the sliding member being connected to the telescopic cylinder, and the driving assembly being mounted within the sliding member to drive the telescopic cylinder to move. The driving assembly comprises a rotating shaft, and the spare assembly comprises: a worm wheel portion mounted on the rotating shaft; and a worm portion mounted at a predetermined distance from the worm wheel portion, the worm portion having a first adjusting portion and a second adjusting portion, the first adjusting portion being operable to engage the worm portion with the worm wheel portion, and the second adjusting portion being operable to drive the worm wheel portion by means of the worm portion, so as to drive the rotating shaft to move.

Some exemplary embodiments have been described above; however, it should be understood that various modifications may be made. For example, suitable results can be achieved if the described techniques are performed in a different order and/or if components in the described systems, architectures, devices, or circuits are combined in different ways and/or replaced or supplemented by additional components or equivalents thereof. Accordingly, other implementations also fall within the scope of the claims.

1. A spare assembly for a medical imaging system, the medical imaging system comprising a driving assembly, the driving assembly comprising a rotating shaft, and the spare assembly comprising:
a worm wheel portion mounted on the rotating shaft; and
a worm portion mounted at a predetermined distance from the worm wheel portion, the worm portion having a first adjusting portion and a second adjusting portion, the first adjusting portion being operable to engage the worm portion with the worm wheel portion, and the second adjusting portion being operable to drive the worm wheel portion by means of the worm portion, so as to drive the rotating shaft to move.

2. The spare assembly according to claim 1, wherein the second adjusting portion is capable of controlling movement of the rotating shaft when the driving assembly fails, thereby driving an assembly connected to the rotating shaft to move.

3. The spare assembly according to claim 1, wherein the worm portion is mounted at a side edge of the worm wheel portion, and the second adjusting portion is located at a bottom portion of the worm portion.

4. The spare assembly according to claim 1, wherein the medical imaging system comprises a suspension apparatus, the suspension apparatus comprises a set of perpendicularly mounted guide rails, a sliding member, and a telescopic cylinder, the guide rails are mounted on a ceiling, the sliding member is mounted on the guide rails, the sliding member is connected to the telescopic cylinder, the driving assembly is mounted within the sliding member to drive the telescopic cylinder to move, and the second adjusting portion of the worm portion is operable to drive the telescopic cylinder to raise or lower.

5. The spare assembly according to claim 4, wherein the driving assembly further comprises a balancing apparatus, and the second adjusting portion of the worm portion is further operable to drive the balancing apparatus to raise or lower.

6. The spare assembly according to claim 5, wherein a top portion of the balancing apparatus is provided with a wire rope fixing portion and a hanging point, the hanging point is provided at a position relating to the center of gravity of the balancing apparatus, and a wire rope can be fixed to the wire rope fixing portion by means of the hanging point, so as to control raising or lowering of the balancing apparatus by means of the second adjusting portion.

7. The spare assembly according to claim 1, further comprising a suspension apparatus comprising a set of perpendicularly mounted guide rails, a sliding member, and a telescopic cylinder, the guide rails being mounted on a ceiling, the sliding member being mounted on the guide rails, the sliding member being connected to the telescopic cylinder, the driving assembly being mounted within the sliding member to drive the telescopic cylinder to move.

8. The spare assembly according to claim 7, wherein the driving assembly comprises:
a drum rotatable about the rotating shaft;
a main wire rope, one end of which is fixed onto the balancing apparatus and the other end of which is fixed onto the telescopic cylinder by means of the drum; and
an auxiliary wire rope, one end of which is fixed onto the drum and the other end of which is fixed onto the telescopic cylinder.

9. The spare assembly according to claim 8, wherein the main wire rope and the auxiliary wire rope are capable of being wound around the drum in an alternating manner, or the main wire rope is wound around one end of the drum and the auxiliary wire rope is wound around the other end of the drum.

10. The spare assembly according to claim 8, wherein the telescopic cylinder comprises a plurality of cylinder shells which are sleeved from top to bottom, wherein a wire rope fixing support is mounted at a top portion of the lowermost cylinder shell, the wire rope fixing support comprises a body portion and a cover plate, the body portion comprises at least one opening for receiving the wire ropes, the cover plate is movable relative to the body portion to cover at least a portion of the opening to fix the positions of the wire ropes.

11. The spare assembly according to claim 10, wherein the driving assembly further comprises a line encoder mounted adjacent to the drum, the line encoder comprising a connecting line, and the other end of the connecting line being fixed to the lowermost cylinder shell.

12. The spare assembly according to claim 8, wherein one end of the drum is provided with a ratchet, and the driving assembly further comprises a safety locking mechanism, the safety locking mechanism being mounted adjacent to the ratchet, and being capable of engaging with the ratchet to lock the rotating shaft when the main wire rope is broken.

13. The spare assembly according to claim 12, wherein the safety locking mechanism comprises a rotating portion and an extension portion, the extension portion is connected to the rotating portion, an end portion of the rotating portion is provided with a gear, and the gear is capable of engaging with the ratchet.
